(19) United States
(12) Patent Application Publication
(10) Pub. No.: US 2018/0338512 A1
(43) Pub. Date: Nov. 29, 2018

(54) FOOD PROCESSING APPLIANCE

(71) Applicant: KENWOOD LIMITED, Havany, Hampshire (GB)

(72) Inventors: Paul PALMER, Havant (GB); Sivaprakash SHANMUGAM, Havant (GB); James Joseph SEALY, Havant (GB); Robert FIELDS, Havant (GB); Bradley CUNNINGHAM, Havant (GB)

(73) Assignees: KENWOOD LIMITED, Havant, Hampshire (GB); KENWOOD LIMITED, Havant, Hampshire (GB)

(21) Appl. No.: 15/755,866

(22) PCT Filed: Aug. 22, 2016
(86) PCT No.: PCT/GB2016/052599

§ 371 Date: Feb. 27, 2018

(30) Foreign Application Priority Data
Aug. 28, 2015 (GB) 1515398.4

(51) Int. Cl. A23L 3/28

(52) U.S. Cl. CPC A23L 3/28

(57) ABSTRACT

A food processing appliance (100) comprising an enclosure (102), (104) for receiving an item; a UV radiation source (204) operable to irradiate items in the enclosure (102), (104) with UV light; and a safety mechanism (208), (402) arranged to prevent access to the enclosure when the radiation source is irradiating the item in the enclosure.

This invention relates food processing appliances. Generally, it relates to kitchen machines, in particular to such machines as are commonly referred to as food mixers, food processors and blenders and the like (food processing appliances/kitchen machines).

Food mixers typically comprise stand mixers, by which is meant the kind of kitchen machine in which a receptacle such as a mixing bowl is supported on a pedestal which also supports an electric motor and a drive system including a drive outlet, overhead of the bowl (hereafter called the container), which permits a planetary mixing action to be imparted to tools suspended into the bowl from the overhead drive outlet.

Food processors, on the other hand, typically comprise machines in which the container equipped with rotatable blades or the like can be driven from beneath by means of a motor and drive system housed within a casing upon which the container is supported. Frequently such food processors present two drive outlets (sometimes these are disposed coaxially) which can selectively drive the blades or the like associated with different containers, such as a bowl and a goblet, for mixing and blending respectively.

Blenders, typically comprise machines in which the container equipped with rotatable blades or the like can be driven from beneath by means of a motor and drive system housed within a casing upon which the container is supported.

These types of such kitchen machines are extremely versatile, and recent technical developments provide the possibility of still broader operational capability. Therefore, these machines are being used more often in the kitchens all around the world. There are two hygiene issues associated with foodstuffs in food processing appliances, one is the cleanliness of the foodstuff and second is the cleanliness of the container before processing. Currently, users clean the food items before placing them in the kitchen machines. Users may clean food items by washing under water. For food items with thick skin, they may use a vegetable brush to help wash away hard-to-remove microbes. Further, for food items with a lot of nooks and crannies like cauliflower or lettuce, the user soaks the food item in cold clean water. However, for fragile food products such as raspberries, users may just spray distilled water on the food items. After washing, users often dry with clean paper towel as this can remove more bacteria. Further, some food washing products are available in the market, which might help remove pesticides, chemical, heavy metals, dirt, and wax from the surface of fruits and vegetables. Often the container may need to be manufactured with what are known in the industry as dirt traps for example near any fixings or sharp corners. Many containers are dish washable and user instructions generally recommend that the bowl is washed in warm soapy water before use. Sometimes, however, the dirt traps retain organic/microbial material which may decompose and cause the spread of bacteria. Therefore there is a need for improved food processing appliance which can assist users in cleaning their food items and/or the containers they are processed in.

It is therefore desirable to provide improved food processing appliance that helps sterilize the food and other items including the container and the blades and accessories of the food mixer. (In this application, “sterilization” includes “disinfection” and does not necessarily require 100% of the microbes present being destroyed, but instead may be satisfied by e.g., ˜99% of the microbes present being killed).

More specifically, the solution to the problems is provided in a way such that it mitigates the existing problems around the implementation of such solutions within the home environment for appliances.

A food processing appliance comprising: an enclosure for receiving an item; a UV radiation source operable to irradiate items in the enclosure with UV light; and a safety mechanism arranged to prevent access to the enclosure when the radiation source is irradiating the item in the enclosure. Preferably, the radiation source is adapted to irradiate substantially the whole of the interior of the enclosure.

Preferably, the enclosure comprises a container with a lid and the safety mechanism comprises an interlock mechanism which allows operation of the UV radiation source only when the lid is attached to the container.

Preferably, the interlock mechanism comprises a push-rod or switch actuated by attachment of the lid so as to connect the UV radiation source to an external power source.

Preferably, the food processing appliance further comprises a processor for controlling the UV radiation source.

Preferably, the food processing appliance further comprises a UV light level sensor in electronic communication with the processor for detecting the UV light level within the container. The processor may be configured to adjust the level of UV light based on feedback from the UV light level sensor.

Preferably, the food processing appliance further comprises a pressure sensor in electronic communication with the processor for detecting the attachment of the lid to the container.

The processor may be configured to only allow activation of the UV radiation source when the pressure sensor detects that the lid is attached.

Preferably, the processor is adapted to execute a sterilization program. The sterilization program may comprise periodic brief high-intensity sterilization to rapidly sterilize the container interspersed with long periods of low-intensity sterilization to maintain a sterile environment. The sterilization program may be activated in response to an event, preferably following the occurrence of an event that might compromise sterility.

Preferably, the food processing appliance further comprises a user interface in communication with the processor whereby the user can select to have the processor execute the sterilization program.

Preferably, the food processing appliance further comprises a base unit on which the container is detachably positioned.

The base unit may include a first relay contact and the container includes a second relay contact such that the first and second relay contacts interact when the container is in positioned on the base unit. One of the relay contacts may be concave and the other convex, such that one relay contact may fit into the other relay contact. The activation of the UV radiation source may be in dependence on the interaction between the first and second relay contacts. Preferably, at least one relay contact is connected to external electricity supply, such that when the relay contacts interact the electric circuit to the UV radiation source is completed.

The processor may be located in either the base unit or the container.

The food processing appliance may be adapted to sterilize by UV irradiation at least one of: items in the enclosure; at least part of the enclosure; and any other parts of the food processing appliance located in the enclosure. The enclosure is adapted to receive an item comprising one or more of: food; liquids; appliance accessories, such as food processing tools for mixing, chopping, stirring and spatula tools; contact lenses; and baby bottles, baby bottle nipples.

Preferably, the UV radiation source is only operational when the appliance is processing food with a processing tool.

Preferably, the enclosure is impervious to UV radiation. More preferably, at least part of the inside surface of the enclosure is coated with a UV reflecting material. Yet more preferably, at least part of the enclosure has an electro-chromic capability such that it can be made opaque to the UV radiation. For example, the electro-chromic ability may be switchable such that at least part of the enclosure is opaque when the UV radiation source is on and clear when the UV radiation source is off. Alternatively, or in addition, the electro-chromic ability may be switchable such that the container is opaque when removed from the base.

Preferably, the container and the lid are made of stainless steel, glass or UV stabilized plastic. The container may be either i) coated in an anti-microbial material and/or ii) manufactured from an anti-microbial material or a plastic containing an anti-microbial material.

Preferably, the UV radiation source is germicidal, preferably emitting radiation of a wavelength in the range 120 nm-450 nm. Preferably, the UV radiation source is adapted to be switched on/off periodically.

Preferably, the UV radiation source is located: in or on the lid, preferably in or on the rim of the lid; in or on a rim of the container; in any surface of the container; distributed around the inside surface of the container; in the bottom of the container; in the base of the container and shone through the container from underneath; placed such that all UV light generated by the UV light source is focused towards any item placed on the bottom of the container; and/or at an area of the container beneath any food processing tool when being used with the appliance;

Preferably, the UV radiation source comprises a UV strip light, preferably a plurality of UV lamps or alternatively LEDs or a plurality of individual UV lamps LEDs.

Preferably, the food processing appliance comprises: a food mixer or stand mixer, in which a receptacle such as a mixing bowl is supported on a pedestal which also supports an electric motor and a drive system including a drive outlet, overhead of the bowl (hereafter called the container), which permits a planetary mixing action to be imparted to tools suspended into the bowl from the overhead drive outlet; or a food processor or blender, in which a container equipped with rotatable blades or the like can be driven from beneath by means of a motor and drive system housed within a casing upon which the container is supported.

Features of the invention may include one or more of:
A food processing appliance (100) comprising a container (102) with a lid (104) for providing an enclosure for a food item. A rim (202) of the container (102) including a UV light source (204). Further, the food mixer (100) includes a base unit (108) to dock the container (102).
A food processing appliance (100) comprising: a container (102) with a lid (104) for providing an enclosure for a food item, a portion (202) of the container (102) including a UV light source (204); and a base unit (108) to dock the container (102).
The UV light source (204) positioned in the lid of the appliance, in any surface of the container, in the base unit of the appliance.
The appliance (100) comprising one or more of a juicer, a blender, a mixer and a grinder.
An electrical connection (206), optionally a wired connection, providing electricity to the UV light source (204).
The container (102) comprising a relay contact (208), wherein the electrical connection (206) connects the UV light source (204) to the relay contact (208).
The base unit (108) including a relay contact (402), wherein the relay contact (208) connects with the relay contact (402), when the container (102) is placed on the base unit (108).
The UV light source (204) being a UV strip light (302), optionally including a plurality of UV LEDs, or including a plurality of individual UV LEDs (304), (306, (308), (310) and (312).
The food processing appliance being adapted to sterilize at least one of the container (102), the blade (106) and a food item in the container (102).
The food processing appliance container made of a plastic containing an anti-microbial material and/or made of an anti-microbial material.

Other technical advantages will be readily apparent to one skilled in the art from the following figures, descriptions and claims. Moreover, while specific advantages have been enumerated above, various embodiments may include all, some or none of the enumerated advantages.

The invention also provides a computer program and a computer program product for carrying out any of the methods described herein, and/or for embodying any of the apparatus features described herein, and a computer readable medium having stored thereon a program for carrying out any of the methods described herein and/or for embodying any of the apparatus features described herein.

The invention also provides a signal embodying a computer program for carrying out any of the methods described herein, and/or for embodying any of the apparatus features described herein, a method of transmitting such a signal, and a computer product having an operating system which supports a computer program for carrying out the methods described herein and/or for embodying any of the apparatus features described herein. The invention extends to methods and/or apparatus substantially as herein described with reference to the accompanying drawings.

Any feature in one aspect of the invention may be applied to other aspects of the invention, in any appropriate combination. In particular, method aspects may be applied apparatus aspects, and vice versa.

Equally, the invention may comprise any feature as described, whether singly or in any appropriate combination.

Furthermore, features implemented in hardware may generally be implemented in software, and vice versa. Any reference to software and hardware features herein should be construed accordingly.

An embodiment of the invention will now be described with reference to the accompanying drawings in which:

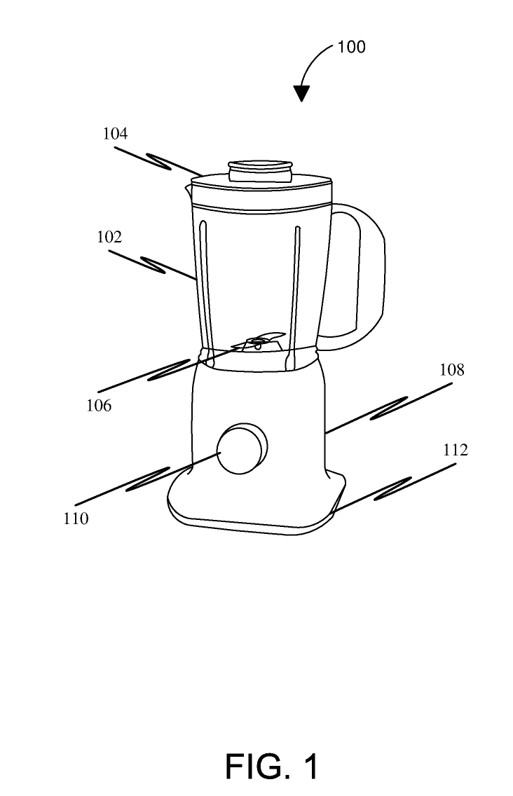

FIG. 1 shows a front view of a food processing appliance;

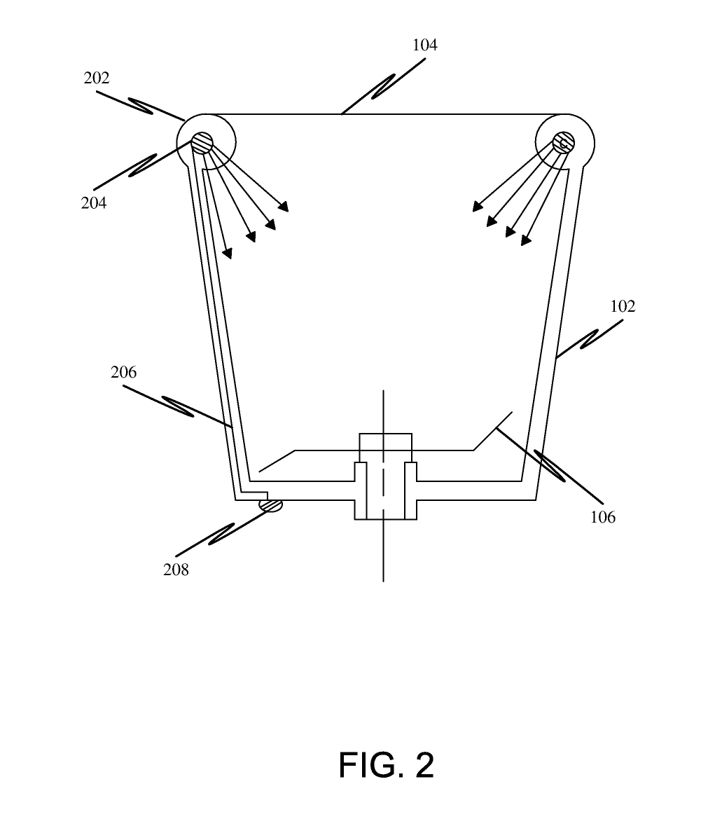

FIG. 2 shows a cross section view of the container of the food processing appliance;

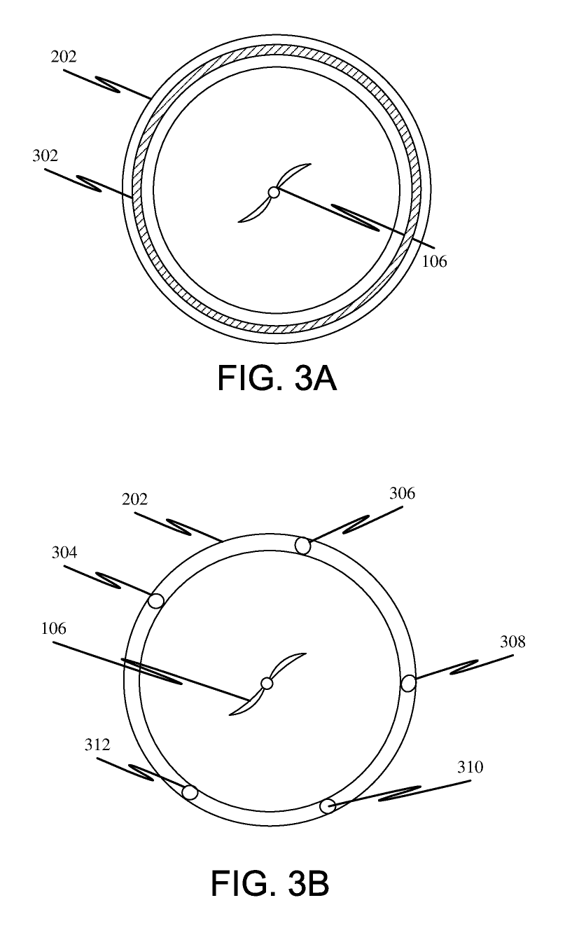

FIG. 3A shows a top view of the container of the food processing appliance;

FIG. 3B shows a top view of the container of the food processing appliance;

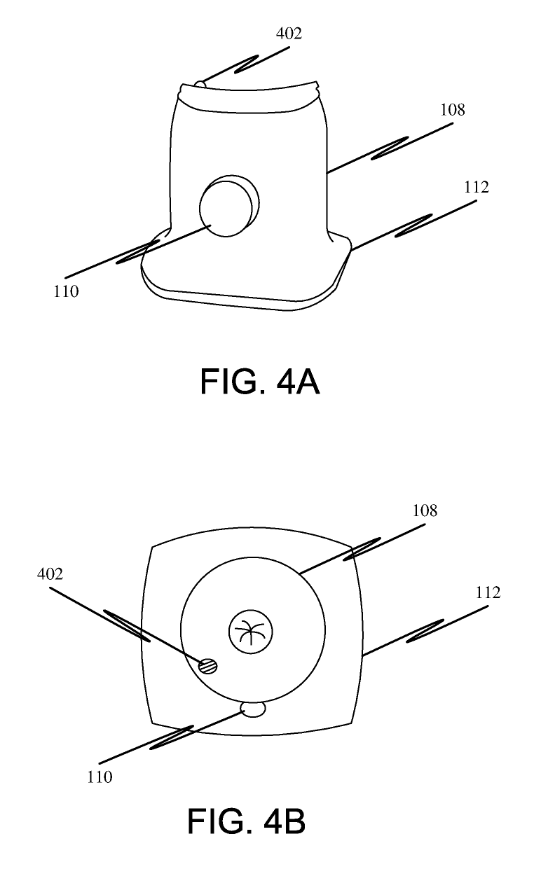

FIG. 4A shows a front view of a base unit of the food processing appliance;

FIG. 4B shows a top view of the base unit of the food processing appliance;

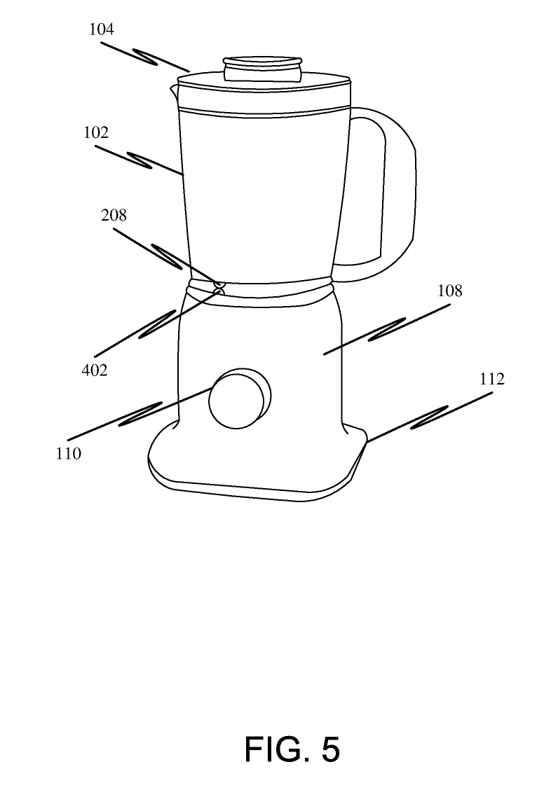

FIG. 5 shows electronic contact points embedded in the food processing appliance;

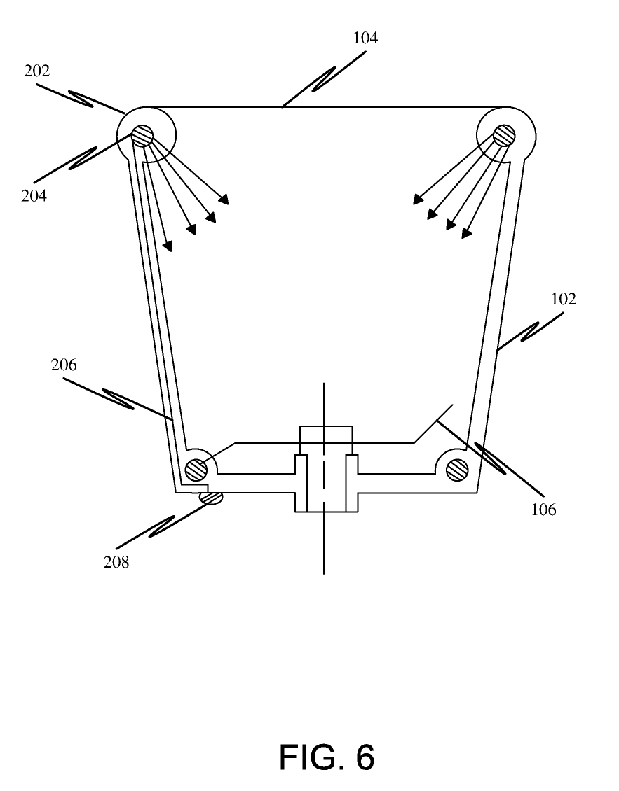

FIG. 6 shows a cross section view of the container of the food processing appliance;

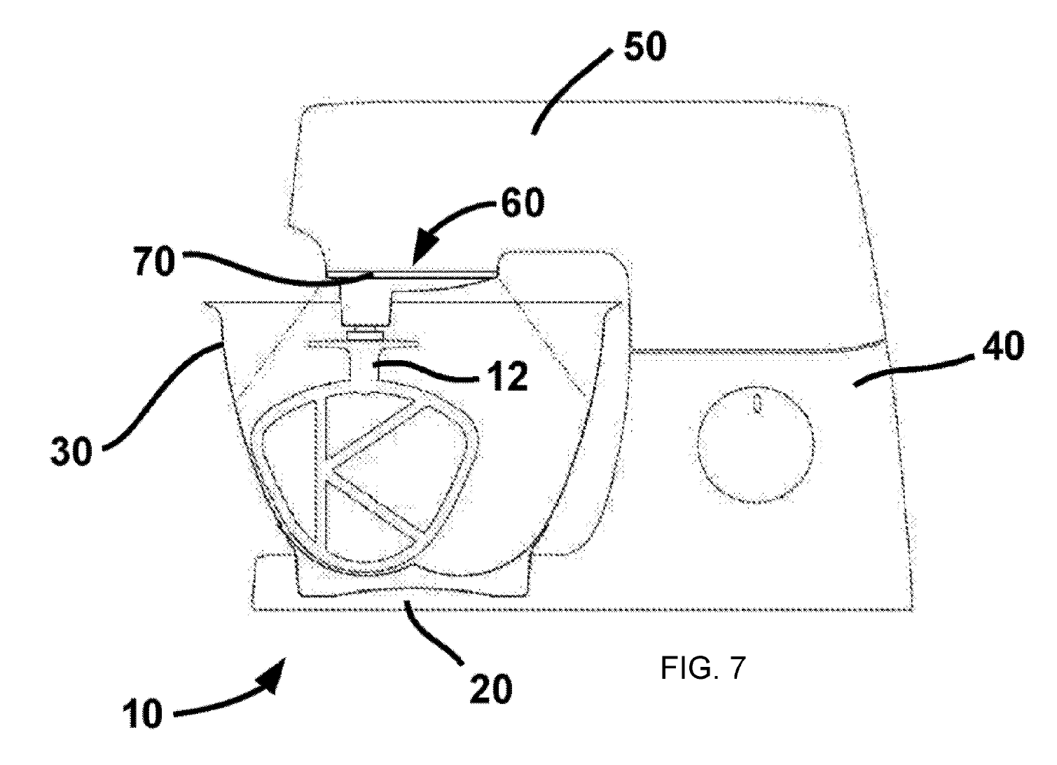

FIG. 7 shows a side-on cut-away view of a food processing appliance according to an embodiment of the invention; and

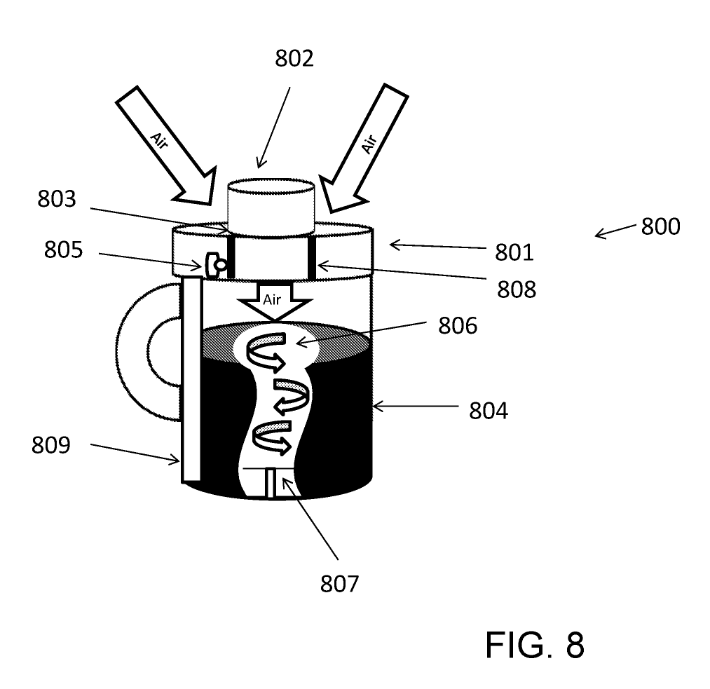

FIG. 8 shows a schematic perspective view of a blender according to another embodiment.

FIG. 1 shows a food mixer (100) comprising a container (102) with a lid (104) for providing an enclosure for a food item. The container (102) also includes a blade (106). The blade (106) may be detachably connected to the container. The food processing appliance (100) further includes a base unit (108) on which the container (102) rests. The base unit (108) includes controls (110) that allow a user to operate the food processing appliance (100) and a UV light source (204). The base unit (108) may also include a support (112) that provides stability to the base unit (108).

FIG. 2 shows a cross section view of the container (102) of the food processing appliance (100). As shown, the rim (202) of the container (102) includes a UV light source (204). The light emitted by the UV light source (204) may be truly UV and preferably germicidal. Also, the UV light source (204) may emit UV radiation with wavelength in the range 120 nm-450 nm. UV light with a wavelength in the range 250-260 nm is particularly effective as a germicide.

FIG. 3 show alternative top views of the container (102) of the food processing appliance. The UV light source (204) may be a UV strip light (302) as shown in FIG. 3A, wherein the UV strip light (302) includes a plurality of UV lamps or LEDs (not shown). Alternatively, the UV light source (204) may include a plurality of individual UV lamps LEDs (304), (306, (308), (310) and (312), as shown in FIG. 3B.

The UV light source (204) is placed such that all UV light generated by the UV light source (204) is focused towards a food item placed on the bottom of the container (102). The light source may equally be situated in the bottom of the container or in any surface of the container or equally in the lid. The UV source (204) may be contained in the base and shone through the container from underneath. To facilitate this, the container may feature windows made of material relatively transparent to UV light, such as polydimethylsiloxane (PDMS) through which the UV light source (204) may shine. Locating the UV light source (204) so that it does not directly contact food is advantageous in that cooling of the UV light source (204) by food (e.g., ice) that may reduce its UV emission is avoided. The container (102) and the lid (104) may be made of stainless steel, glass or UV stabilized plastic. The container (102) and the lid (104) do not allow UV light to escape; thereby avoiding exposing users to any UV radiation. Further, the inside surface of the container (102) may be coated with a UV reflecting material such as aluminium, or the container may have an electro-chromic capability and hence be made opaque to the UV when the UV light source (204) is on, such that all UV radiation stays inside the container (102). Making the blade (106) and container (102) of a highly UV-reflective material such as aluminium is advantageous as reflection of UV light will allow it to reach within corners and recesses to ensure sterilisation of areas not within direct line-of-sight of the UV light source (204). Further, the electro-chromic ability may be switchable such that the container material may be opaque when the UV light source (204) is on and clear when the UV light source (204) is off and on when the container is removed from the base to facilitate storage and the prevention of UV radiation entering the processed foodstuff which may have a detrimental effect. Further, the UV light source (204) may be switched on/off periodically to prevent harmful exposure to UV radiation. Yet further, the UV light source (204) is switched on only when the blade (106) is operational. This will ensure that food placed in the container (102) is uniformly sterilized.

Further, the container (102) includes an electrical connection (206) connecting the UV light source (204) and to an electrical connection (208) embedded in the base of the container (102). The electrical connection (206) may be a metal wire extending from the UV light source (204) to the electrical connection (208). The electrical connection (208) may be a relay contact.

As show in FIGS. 4A and 4B, the base unit (108) includes a relay contact (402) on the top surface where the container sits on the base unit (108). The relay contact (208) may be placed in any suitable location in the container (102) such that when the container (102) is placed on the base unit (108), the relay contact (208) comes in contact with the relay contact (402) placed on the base unit (108) (as shown in FIG. 5). Further, the shapes of the relay contact (208) and the relay contact (402) may vary. For example, one of the relay contacts may be concave and the other convex, so that one relay point or contact fits into the other relay point or contact. The relay contact (402) is connected to external electricity supply. So, when the relay contact (208) comes in contact with the relay contact (402), the electric circuit to the UV light source (204) is complete.

The base unit (108) may include a PCB (not shown) having processor and memory resources for controlling the UV light source (204) and other elements in the container. The PCB may alternatively be located in the container (102). The container (102) may include one or more UV light sensors (not shown) in electronic communication with the PCB, for detecting the UV light level within the container and allowing the adjusting of the level of UV light by the PCB based on feedback from the UV light level sensors. In this way the PCB may adapt the UV light emitted by the UV light source (204) for differing ambient UV light levels created by, e.g., differing levels of sunshine or other external UV light sources.

The base unit (108) may further include a user interface (not shown) having a display and buttons for selecting options on the display, whereby the user can select to execute a sterilization program stored on the memory of the PCB, which the PCB, which is in bi-directional electronic communication with the user interface, then carries out. Examples of sterilization programs include periodic brief high-intensity sterilization to rapidly sterilize the container interspersed with long periods of low-intensity sterilization to maintain a sterile environment; and sterilization in response to an event (e.g., energizing of a motor of the food mixer (100) to drive the blade (106), or attachment of the lid (104) to the container (102)) detected by the PCB using suitable sensor means to ensure a sterile environment following the occurrence of an event that might compromise sterility.

To prevent accidental exposure of the user to UV light emitted by the UV light source (204), the UV light source (204) may be interlocked so that it can only be switched on when the lid (104) is attached to the container (102). This can be achieved by the PCB being configured to only activate the UV light source (204) when a sensor (e.g., a pressure-switch) detects that the lid (104) is attached. Interlocking of the UV light source (204) may also be achieved by a push-rod or switch (not shown) being actuated by attachment of the lid (104) so as to connect the UV light source (204) to an external power source.

Whilst the UV light source (204) is described as being located on a rim (202) of the container (102), the UV light source (204) may be located at different locations. For example, the UV light source (204) may be located at an area of the container beneath the blade (106) to ensure sterilization of the under-side of the blade, and/or the UV light source (204) may be distributed around the inside surface of the container to ensure even sterilization.

FIG. 7 shows a stand mixer (10) according to an embodiment of the invention. The stand mixer (10) includes a rotary tool (12) depending from the head (50) into the bowl (30) which rests on the pedestal (20). The rotary tool (12) may be driven by a motor located either in the upright section (40) or in the head section (50), and is surrounded by a UV light source (70) such that the rotary tool (12) and the contents of the bowl (20) are bathed in UV light (denoted by rays in FIG. 7) regardless of the rotary position of the rotary tool (12). Upright section (40) includes a control knob whereby the speed of the motor (and thus the rotary tool (12)) may be varied during operation. The intensity of the UV light emitted by the UV light source (70) may be controlled by a CPU located in the stand mixer (10) to vary with the speed of the motor. For example, the intensity of UV light may increase as the speed of the motor increases to ensure sterilisation of the food. The UV light source (70) may be a strip-light (e.g., fluorescent tube) or a series of UV light sources (e.g., LEDs) located behind a UV light-diffuser (e.g., a panel of surface-roughened PDMS) so as to create a pseudo-strip-light-like effect. The invention may also be implemented in a wand-like hand-blender or hand-mixer, with the UV light source similarly surrounding the axis of rotation of the tool(s) in a ring/oval-like fashion.

Further, the food processing appliance (100) sterilizes the container (102) and the blade (106), along with the food items in the container (102).

FIG. 8 illustrates a further embodiment in which UV sterilisation is implemented in a blender 800 with a receptacle 804 having a lid 801 with an (optionally removable) filler-cap 802 located in a central aperture of the lid 801 through which (when the filler cap 802 is removed) ingredients may be inserted into the receptacle 804. Due to the potential expansion/compression of air within the receptacle 804 it is necessary to have an air channel through which air can travel into and out of the receptacle 804 (as indicated by the large arrows), therefore one or more air channels 803 are formed by the filler cap 802. The air channel 803 may be formed e.g., as a gap between the filler cap 802 and the lid 801 as shown in FIG. 8, or may be a through-hole defined through the filler cap 802, or indeed as a through-hole defined through or between the lid 801 and receptacle 804.

Unfortunately air includes microbes as well as oxygen, the presence of which allows microbial growth within material being processed once drawn into it by the vortex 806 created by the action of processing tool 807, or otherwise brought into contact with the material being processed. To sterilise air flowing through the air channel, therefore, one or more UV-emitting lights 805, such as UV LEDs, emits UV light (e.g., UVC light) across the air channel 803, thus sterilising the air flow. The UV light emitted by the UV lights 805 may be intensified by coating or constructing the sides of the air channel 808 with UV-reflecting material such as aluminium. To prevent harmful levels of UV light escaping from the air channel 803, the portions of the sides of the air channel 808 close to the entrances to the air channel 803 may be coated or constructed of a UV-absorbing material (e.g., a UV-absorbent plastic) and/or form a dog-leg (with e.g. baffles formed within it) so that there is no straight-line passage from the UV lights 805 out of the air channel 803.

With the above configuration, high levels of UV light such as may rapidly sterilise air (or any other material inserted into the receptacle 804 through the air channel 803, including food and water) flowing through the air channel 803 at the speeds typical of blender operation may be used without causing harmful levels of UV light to escape the air channel 803 which might harm the user or the material being processed. Receptacle 804 may include additional UV lights transmitting at lower intensity within it to simultaneously maintain a sterile environment within it without harming the material being processed, alternatively UV light from the UV lights 805 may be reflected into the receptacle 804 by, for example, the sides of the air channel 808 or by another reflector, with the reflector being made partially absorbent so as to reduce the intensity of the UV light to a safe level before reflecting the light into the receptacle 804 to slowly sterilise the material being processed.

Blender 800 may include a cable enclosure 809 through which power and data may be conveyed to/from the base of the blender 800 to the lid 801. In this way the UV lights 805 may receive power from the base (which in turn may be mains-powered) and data from sensors (e.g., pressure switches indicating the presence/absence of the lid 801, filler cap 802, or material to be processed) may be conveyed to a processor in the base of the blender 800 to allow the processor to control the UV lights 805 based on the data received from the sensors. For example, the processor may turn on the UV lights 805 when the lid 801 and/or filler cap 802 are present, or when material to be added is not present (so that the receptacle is thoroughly sterilised before food is placed in it).

In further embodiments, the food processing appliance (100) may be adapted for sterilizing contact lenses, bottles, baby bottle nipples, liquids and the appliance accessories such as mixing, chopping, stirring and spatula tools for example.

In further embodiments the container may be coated in an anti-microbial material.

In further embodiments the container may be manufactured from a plastic containing an anti-microbial material.

In further embodiments the container may be manufactured from an anti-microbial material.

Further modifications will be apparent to those skilled in the art.

Reference numerals appearing in any claims are by way of illustration only and shall have no limiting effect on the scope of the claims.

Where a bottom-driven food processing machine (e.g., a blender) and top-driven food processing machines (e.g., a stand-mixer or kitchen machine) have been used as examples, the invention may instead be implemented in a machine where the rotary tool is initially oriented horizontally or at another angle. Whilst table-top domestic food processing machines have been used as an example implementation, the invention may also be implemented in a hand-held tool such as a hand-blender or hand-blender attachment. The term “kitchen appliance” encompasses all such devices.

Whilst the invention has been described in the field of domestic food processing and preparation machines, it can also be implemented in any field of use where efficient, effective and convenient preparation and/or processing of material is desired, either on an industrial scale and/or in small amounts. The field of use includes the preparation and/or processing of: chemicals; pharmaceuticals; paints; building materials; clothing materials; agricultural and/or veterinary feeds and/or treatments, including fertilisers, grain and other agricultural and/or veterinary products; oils; fuels; dyes; cosmetics; plastics; tars; finishes; waxes; varnishes; beverages; medical and/or biological research materials; solders; alloys; effluent; and/or other substances. Mechanical and other improvements disclosed herein may find application in automotive and industrial fields, the field of tools including hand-tools, the plumbing field, the field of hydraulics, and are not limited in application to kitchen appliances.

The invention described here may be used in any kitchen appliance and/or as a standalone device. This includes any domestic food-processing and/or preparation machine, including both top-driven machines (e.g., stand-mixers) and bottom-driven machines (e.g., food processors). It may be implemented in heated and/or cooled machines. The invention may also be implemented in both hand-held (e.g., hand blenders) and table-top (e.g., blenders) machines. It may be used in a machine that is built-in to a work-top or work surface, or in a stand-alone device. The invention can also be implemented as a stand-alone device, whether motor-driven or manually powered.

It will be understood that the invention has been described above purely by way of example, and modifications of detail can be made within the scope of the invention.

Each feature disclosed in the description, and (where appropriate) the claims and drawings may be provided independently or in any appropriate combination.

Reference numerals appearing in any claims are by way of illustration only and shall have no limiting effect on the scope of the claims.

1.-40. (canceled)

41. A food processing appliance comprising:
an enclosure for receiving an item;
a UV radiation source operable to irradiate items in the enclosure with UV light; and
a safety mechanism arranged to prevent access to the enclosure when the radiation source is irradiating the item in the enclosure, and
a processor for controlling the UV radiation source.

42. A food processing appliance according to claim 41, wherein the enclosure comprises a container with a lid and the safety mechanism comprises an interlock mechanism which allows operation of the UV radiation source only when the lid is attached to the container, preferably wherein the interlock mechanism comprises a push-rod or switch actuated by attachment of the lid so as to connect the UV radiation source to an external power source.

43. A food processing appliance according to claim 41, further comprising a UV light level sensor in electronic communication with the processor for detecting the UV light level within the container, preferably wherein the processor is configured to adjust the level of UV light based on feedback from the UV light level sensor.

44. A food processing appliance according to claim 42, further comprising a sensor, preferably a pressure sensor in electronic communication with the processor for detecting the attachment of the lid to the container, preferably wherein the processor is configured to only allow activation of the UV radiation source when the pressure sensor detects that the lid is attached.

45. A food processing appliance according to claim 41, wherein the processor is adapted to execute a sterilization program, and preferably wherein the sterilization program comprises periodic brief high-intensity sterilization to rapidly sterilize the container interspersed with long periods of low-intensity sterilization to maintain a sterile environment.

46. A food processing appliance according to claim 45, wherein the sterilization program is activated in response to an event, preferably following the occurrence of an event that might compromise sterility, and/or further comprising a user interface in communication with the processor whereby the user can select to have the processor execute the sterilization program.

47. A food processing appliance according to claim 42, wherein the appliance comprises a base unit on which the container is detachably positioned, and/or wherein the processor is located in either the base unit or the container.

48. A food processing appliance according to claim 47, wherein the base unit includes a first relay contact and the container includes a second relay contact such that the first and second relay contacts interact when the container is in positioned on the base unit, for example wherein one of the relay contacts is concave and the other is convex, such that one relay contact fits into the other relay contact, and preferably wherein the activation of the UV radiation source is in dependence on the interaction between the first and second relay contacts, for example wherein at least one relay contact is connected to external electricity supply, such that when the relay contacts interact the electric circuit to the UV radiation source is completed.

49. A food processing appliance according to claim 41, adapted to sterilize by UV irradiation at least one of:
i) items in the enclosure;
ii) at least part of the enclosure; and
iii) any other parts of the food processing appliance located in the enclosure.

50. A food processing appliance according to claim 41, wherein the enclosure is adapted to receive an item comprising one or more of:
i) food;
ii) liquids;
iii) appliance accessories, such as food processing tools for mixing, chopping, stirring and spatula tools;
iv) contact lenses; and
v) baby bottles, baby bottle nipples.

51. A food processing appliance according to claim 50, wherein the UV radiation source is only operational when the appliance is processing food with a processing tool.

52. A food processing appliance according to claim 41, wherein the enclosure is impervious to UV radiation, and/or wherein at least part of the inside surface of the enclosure is coated with a UV reflecting material.

53. A food processing appliance according to claim 52 wherein at least part of the enclosure has an electro-chromic capability such that it can be made opaque to the UV radiation, and preferably wherein the electro-chromic ability is switchable such that at least part of the enclosure is opaque when the UV radiation source is on and clear when the UV radiation source is off, for example wherein the electro-chromic ability is switchable such that the container is opaque when removed from the base.

54. A food processing appliance according to claim 42, wherein the container and the lid are made of stainless steel, glass or UV stabilized plastic, and/or wherein the container is either i) coated in an anti-microbial material and/or ii) manufactured from an anti-microbial material or a plastic containing an anti-microbial material.

55. A food processing appliance according to claim 41, wherein the UV radiation source is germicidal, preferably emitting radiation of a wavelength in the range 120 nm-450 nm, and/or wherein the UV radiation source is adapted to be switched on/off periodically.

56. A food processing appliance according to claim 42, wherein the UV radiation source is located:
i) in or on the lid, preferably in or on the rim of the lid;
ii) in or on a rim of the container;
iii) in any surface of the container;
iv) distributed around the inside surface of the container;
v) in the bottom of the container;
vi) in the base of the container and shone through the container from underneath;
vii) placed such that all UV light generated by the UV light source is focused towards any item placed on the bottom of the container; and/or
viii) at an area of the container beneath any food processing tool when being used with the appliance;

57. A food processing appliance according to claim 41, wherein the UV radiation source comprises a UV strip light, preferably a plurality of UV lamps or alternatively LEDs or a plurality of individual UV lamps LEDs.

58. A food processing appliance according to claim 41, comprising:
i) a food mixer or stand mixer, in which a receptacle such as a mixing bowl is supported on a pedestal which also supports an electric motor and a drive system including a drive outlet, overhead of the bowl (hereafter called the container), which permits a planetary mixing action to be imparted to tools suspended into the bowl from the overhead drive outlet in which the UV radiation source is preferably arranged around the tool; or
ii) a food processor or blender, in which a container equipped with rotatable blades or the like can be driven from beneath by means of a motor and drive system housed within a casing upon which the container is supported.

59. A food processing appliance according to claim 41, in which the enclosure comprises at least one channel for allowing air to travel into and out of the enclosure and/or for allowing material to be added into the enclosure, and in which the UV radiation source is arranged to emit UV radiation across the or each channel, and preferably in which the at least one channel is at least partially formed of UV reflective material, and preferably in which a part of the at least one channel adjacent the outside of the enclosure is formed of a UV absorbent material and/or does not provide a straight path through the channel, for example in which the enclosure is arranged such that part of the UV radiation is reflected into the enclosure to irradiate items in the enclosure at a lower intensity than that provided across the channel(s).

60. A food processing appliance according to claim 59, in which the enclosure comprises a conduit for a cable for conveying power and/or data between a base of the appliance and the UV radiation source, and/or in which the at least one channel and/or the UV source is provided in a lid of the enclosure.
